(19) United States
(12) Patent Application Publication
(10) Pub. No.: US 2025/0302287 A1
(43) Pub. Date: Oct. 2, 2025

(54) Device For Estimating Glucose Concentration

(71) Applicant: Taiyo Yuden Co., Ltd., Tokyo (JP)

(72) Inventor: Katsuhiro OYAMA, TOKYO (JP)

(21) Appl. No.: 19/091,986

(22) Filed: Mar. 27, 2025

(30) Foreign Application Priority Data
Mar. 28, 2024 (JP) 2024-053351

(51) Int. Cl. A61B 1/06

(52) U.S. Cl. CPC A61B 1/0638

(57) ABSTRACT

A glucose concentration estimating device includes a first light source, a second light source, a third light source, a light receiving element and an estimating unit. The first light source irradiates a living body with first light which includes one wavelength in a wavelength range of 1375-1395 nm. The second light source irradiates the living body with second light which includes one wavelength in a wavelength range of 1575-1595 nm. The third light source irradiates the living body with third light which includes one wavelength in a wavelength range of 1835-1855 nm. The light receiving element receives reflected light returning from the living body upon irradiating the living body with the first to third light. The estimating unit estimates a glucose concentration based on an output of the light receiving element.

### CROSS REFERENCE TO RELATED APPLICATIONS/INCORPORATION BY REFERENCE

This application makes reference to, claims a priority to, and claims benefit from Japanese Patent Application No. JP2024-053351, filed on Mar. 28, 2024.

### FIELD OF THE DISCLOSURE

This disclosure relates to a glucose concentration estimating device that uses an optical detecting unit to estimate a glucose concentration.

### DESCRIPTION OF THE RELATED ART

Conventionally, medical devices and healthcare products have relied on blood sampling to test for blood components such as glucose concentration. Recently, a method of non-invasive detection that uses an optical detector has attracted attention in order to avoid concerns about physical burden on a patient, an infectious disease, and the like.

Japanese Patent No. 6415606 discloses a device in which a laser beam of mid-infrared light having a wavelength of 9.26 μm is generated by an optical parametric oscillator (OPO) that uses excitation light of near-infrared light having a wavelength of 1.06 μm generated by a light source. The generated laser beam is locally emitted (directed) onto a biological epithelium of a test subject (person), and the diffusely reflected light is detected by a photodetector. The device of Japanese Patent No. 6415606 calculates the glucose concentration in the interstitial fluid by using the normalized light intensity that is calculated from a signal ratio between a monitor photodetector and the photodetector. Glucose has high absorption sensitivity in the wavelength region of mid-infrared light having a wavelength of 9.26 μm, and highly accurate detection can be expected in glucose concentration detection.

JP 2011-62335A discloses a blood glucose level monitoring device that includes a reference blood glucose level measuring unit for invasively measuring a reference blood glucose level, a blood glucose level estimating unit for non-invasively estimating a blood glucose level using near-infrared light, and a calibration unit for automatically calibrating the estimated blood glucose level, which is estimated by the blood glucose level estimating unit, by using the reference blood glucose level.

JP 2000-131322A discloses a device for quantifying a concentration of glucose in a living tissue or a body fluid by using absorption of light in the near-infrared region (a wavelength range of 1300 nm to 1900 nm). The device selects at least one wavelength or one wavelength range from each of four wavelength ranges (i.e., from a wavelength range of 1530 nm to 1560 nm, a wavelength range of 1580 nm to 1640 nm, a wavelength range of 1640 nm to 1720 nm, and a wavelength range from 1720 nm to 1750 nm). Then, the device quantifies the glucose concentration based on an absorption signal obtained by using the selected wavelengths (at least four wavelengths) or the selected wavelength ranges (at least four wavelength ranges). The device of JP 2000-131322A selects at least one wavelength or one wavelength range from the 1580 nm to 1640 nm wavelength range as the specific absorbance wavelength for glucose. In addition, the device of JP 2000-131322A uses the absorption signals of wavelengths in the remaining three wavelength ranges to eliminate disturbances caused by biological components other than glucose that are superimposed on the absorption spectra of glucose in the selected one wavelength (or the selected one wavelength range).

### SUMMARY OF THE DISCLOSURE

In the device of Japanese Patent No. 6415606, excitation light of near-infrared light is generated by a YAG light source, but the YAG light source is expensive. Further, an optical system that can be used for the wavelength of 9.26 μm is made from a chemical compound-based material, and this is also expensive. Therefore, a manufacturing cost of the device is high.

The device of JP 2011-62335A may reduce the cost of the device by using the near-infrared light. However, the wavelength of near-infrared light has a strong absorption reaction to the moisture in the human body. Therefore, the device of JP 2011-62335A cannot distinguish the glucose concentration from the moisture concentration based on the detection result with the near-infrared light. In other words, it is necessary to calibrate the estimation result of the blood glucose value if the device of JP 2011-623335A is used. This calibration process uses a detection result of the blood glucose value obtained by an invasive method (blood sampling).

The device of JP 2000-131322A removes the superimposition of “unnecessary components” other than the glucose concentration. In the wavelength range specified in JP 2000-131322A, however, it is impossible to perform, with high accuracy, the concentration determination of each of the unnecessary components.

An object of the present disclosure is to provide a glucose concentration estimating device capable of estimating a glucose concentration at a low cost and with high accuracy by using near-infrared light.

Additional or separate features and advantages of the disclosure will be set forth in the descriptions that follow and in part will be apparent from the description, or may be learned by practice of the disclosure. The objectives and other advantages of the disclosure will be realized and attained by the structure particularly pointed out in the written description and claims thereof as well as the appended drawings.

To achieve these and other advantages and in accordance with the objective of the present disclosure, as embodied and broadly described, in one aspect, the present disclosure provides a glucose concentration estimating device that includes a first light source, a second light source and a third light source. The first light source irradiates a living body with a first light which includes any one wavelength in a wavelength range of 1375 nm to 1395 nm. The second light source irradiates the living body with a second light which includes any one wavelength in a wavelength range of 1575 nm to 1595 nm. The third light source irradiates the living body with a third light which includes any one wavelength in a wavelength range of 1835 nm to 1855 nm. The glucose concentration estimating device also includes a light receiving element for receiving reflected light that returns from the living body upon irradiating the living body with the first light, the second light and the third light. The glucose concentration estimating device also includes an estimating unit that estimates a glucose concentration based on an output of the light receiving element.

The glucose concentration estimating device may further include a fourth light source for irradiating the living body with a fourth light that includes one wavelength in a wavelength range of 2175 nm to 2255 nm. The light receiving element may be configured to be capable of receiving reflected light that returns from the living body upon irradiating the living body with the first light, the second light, the third light and the fourth light.

The glucose concentration estimating device may further include a storage unit that stores estimation information (information for estimation), and the estimation information may include a learned model that is obtained by learning in advance a relationship between information on the reflected light and glucose concentration. The estimating unit may take, as an estimated value of the glucose concentration, a glucose concentration that corresponds to the output of the light receiving element by using the estimation information stored in the storage unit.

The learned model may be a model in which learning is performed on a plurality of combinations of concentrations, and each of the combinations of concentrations may include a glucose concentration, a moisture concentration, and other components' concentration in a dermis layer set for an artificial skin.

The glucose concentration estimating device may further include an attribute information acquisition unit that acquires attribute information regarding an attribute of a test subject. The learned model may be a model in which relationship among the attribution information, information on the reflected light and the glucose concentration is learned based on the attribution information of the test subject, the information on the reflected light returning from a skin of the test subject and a measured value of the glucose concentration of the test subject. The estimating unit may use the estimation information to estimate the glucose concentration of the test subject from the attribute information of the test subject acquired by the attribute information acquisition unit and the output of the light receiving element corresponding to the test subject.

The glucose concentration estimating device may further include a storage unit that stores estimation information. The estimation information may include information that indicates a relationship between the information on the reflected light and the glucose concentration. The estimation information may be generated by using a learned model that is obtained by learning in advance a relationship between the information of the reflected light and the glucose concentration. The estimating unit may take, as an estimated value of the glucose concentration, a glucose concentration that corresponds to the output of the light receiving element by using the estimation information stored in the storage unit.

The learned model may be a model in which learning is performed on a plurality of combinations of concentrations, and each of the combinations of concentrations may include a glucose concentration, a moisture concentration, and other components' concentration in a dermis layer set for an artificial skin.

The glucose concentration estimating device may further include an attribute information acquisition unit that acquires attribute information regarding an attribute of a test subject. The learned model may be a model in which relationship between information on the reflected light and the glucose concentration is learned based on the information on the reflected light returning from a skin of the test subject and a measured value of the glucose concentration of the test subject for every test subject. The information that indicates a relationship between the information on the reflected light and the glucose concentration may indicate relationship among attribute information of the test subject, the information on the reflected light of the test subject corresponding to the attribute information, and the glucose concentration of the test subject corresponding to the attribute information. The estimating unit may use the estimation information to estimate the glucose concentration of the test subject by using the attribute information of the test subject acquired by the attribute information acquisition unit and the output of the light receiving element corresponding to the test subject.

According to the present disclosure, it is possible to estimate the glucose concentration at a low cost and with high accuracy by using near-infrared light of a plurality of types of wavelengths having a relatively large difference in absorbance between glucose and moisture.

### BRIEF DESCRIPTION OF THE DRAWINGS

FIG. 1 schematically shows a mixture of glucose and moisture in a dermis layer of a human body together with irradiation and reflection of near-infrared light.

FIG. 2 is a graph showing a relationship among detection accuracy, a change in glucose, a change in moisture, and a change in the thickness of an epidermis layer when near-infrared light of the wavelength 1550 nm is incident to the human body.

FIG. 3 shows light absorption spectra of glucose and light absorption spectra of moisture.

FIG. 4 shows a schematic configuration of a glucose concentration estimating device according to a first embodiment of the disclosure.

FIG. 5A is a top view of a trapezoidal prism and four triangular prisms associated with the trapezoidal prism in the first embodiment.

FIG. 5B shows an arrangement of the trapezoidal prism, the four triangular prisms, and four light sources.

FIG. 6A shows incidence of first light to a first triangular prism and incidence of third light to a third triangular prism.

FIG. 6B shows incidence of second light to a second triangular prism and incidence of fourth light to a fourth triangular prism.

FIG. 7 is a graph showing a relationship between wavelengths and absorbances for an artificial skin in which glucose, moisture and nanocellulose are present in a mixed condition while altering concentrations of the glucose, the moisture and the nanocellulose.

FIG. 8A is a table to show a relationship between detected values of the reflected light and measured values of glucose concentration for two test subjects.

FIG. 8B is a diagram graphically showing the table of FIG. 8A.

FIG. 9 is a flowchart illustrating a glucose concentration estimating process according to the first embodiment.

FIG. 10 shows a relationship between true values and estimated values when detected values of reflected light obtained upon emitting light of four wavelengths to the human body are used.

FIG. 11 shows a part of a schematic configuration of a glucose concentration estimating device according to a second embodiment of the disclosure.

FIG. 12 is a flowchart illustrating a glucose concentration estimating process according to the second embodiment.

FIG. 13 shows a relationship between true values and estimated values when detected values of reflected light obtained upon emitting light of three wavelengths to the human body are used.

### DETAILED DESCRIPTION OF THE DISCLOSURE

The following is a detailed description of embodiments of the disclosure with reference to the accompanying drawings. The following embodiments are not intended to limit the disclosure, and not all of the combinations of features described in the embodiments are essential for the configuration of the disclosure. The configuration of the embodiments may be modified or changed if necessary depending on the specifications of the device to which the disclosure is applied and various conditions (conditions of use, environment of use, etc.).

The technical scope of the disclosure is defined by the claims and is not limited by the following individual embodiments. The drawings used in the following description may differ in scale and shape from the actual structure in order to make each configuration easier to understand. Parts, elements, and components shown in one of the drawings may be referred to in the description of other drawings.

### First Embodiment

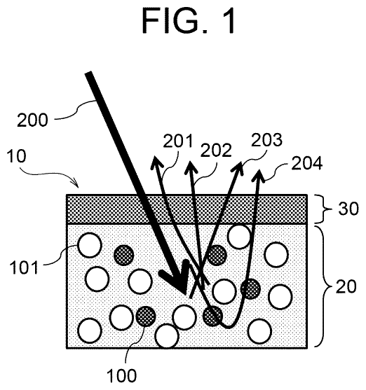

A first embodiment of the present disclosure will be described with reference to FIG. 1 to FIG. 6.

A glucose concentration estimating device 1 of the first embodiment irradiates epidermis of a human body (living body) with a plurality of near-infrared light having a plurality of predetermined wavelengths respectively, and estimates the concentration of glucose in the blood based on the intensity of each light returning from the epidermis of the human body upon the irradiation of the light of each wavelength.

### Reflection of Incident Light at a Dermis Layer

FIG. 1 schematically shows how glucose 100 and moisture 101 are present and mixed in a dermis layer 20 of a human body 10, and also schematically shows an irradiation and reflection of near-infrared light 200.

The light irradiated here 200 is, for example, near-infrared light in a wavelength range from 1300 nm to 2400 nm. The outermost skin of the human body 10 is the epidermis layer 30. The dermis layer 20 is beneath the epidermis layer 30. For a forearm of the human body 10, the thickness of the epidermis layer 30 is approximately 0.2 mm and the thickness of the dermis layer 20 is approximately 2 mm.

As shown in FIG. 1, the glucose 100 and the moisture 101 are mixed in the dermis layer 20. Therefore, when the living body (human body) 10 is irradiated with the light 200, a part of the light 200 is reflected on the surface of the epidermis layer 30, but the remaining light enters the epidermis layer 30. In addition, some of the light 200 that enters the epidermis layer 30 is reflected in the epidermis layer 30 and returns to the outside (proceeds out of the living body 10).

The remainder of the light 200 entering the epidermis layer 30 penetrates the epidermis layer 30 and reaches the dermis layer 20. Some of the light 200 that enters the dermis layer 20 reflects in the dermis layer 20 and proceeds out of the living body 10, as the reflected light 201, 202, 203, and 204 in FIG. 1. The reflected light 201 to 204 is light after light absorption has taken place by the moisture 101, the glucose 100, and/or the like in the dermis layer 20. Thus, the intensity of the incident light 200 and the intensity of the reflected light 201-204 provide, in combination, the absorbance of the reflected light 201-204. As will be described later, since the glucose 100 and the moisture 101 have different absorbances with respect to the wavelength of light, the concentration of glucose can be estimated from the absorbances of reflected light 201 to 204. Therefore, the principle of estimating the glucose concentration in a situation where the glucose 100 and the water 101 are mixed is described with reference to FIG. 1. In reality, the dermis layer 20 contains components that are different from the glucose 100 and the moisture 101 and these components absorb light. For example, tolazamide and triglyceride are known to be contained in the dermis layer 20. Therefore, in order to improve the estimation accuracy of the glucose concentration, it is necessary to consider these components (e.g., tolazamide and triglyceride) in addition to the glucose 100 and the moisture 101.

### Detection Sensitivity to Changes in the Living Body

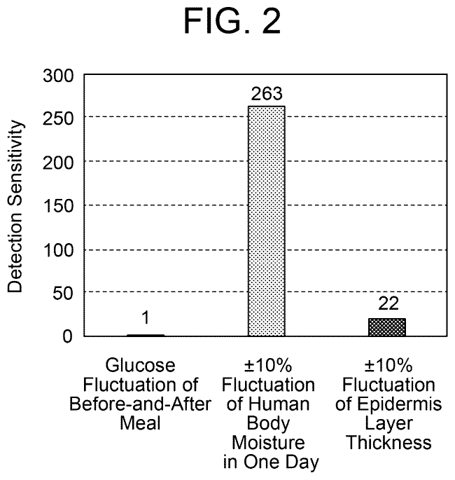

FIG. 2 is a graph showing the relationship between the detection sensitivity and changes in the living body 10 when near-infrared light of the wavelength 1550 nm is used as the incident light 200. The changes in the living body 10 include the change in glucose, the change in moisture content, and the change in the thickness of the epidermis layer 30. In FIG. 2, the horizontal axis represents fluctuation of glucose, fluctuation of moisture, and fluctuation of thickness of the epidermis layer 30. The vertical axis represents detection sensitivity.

The detection sensitivity is based on the absorbance before the human body 10 takes meals (before-meal absorbance), and is calculated from the difference between the before-meal absorbance and the after-meal absorbance. This difference is used as a reference value. The change in glucose before and after the human body 30 takes meal is generally 70 mg/dl of glucose increase. After measuring the absorbance of the artificial skin (before-meal absorbance), the absorbance at the time of 70 mg/dl glucose addition to the artificial skin (after-meal absorbance) was measured to obtain a difference between the two absorbances, and the difference between the two absorbances was used as a reference value for the detection sensitivity.

As shown in FIG. 2, the detection sensitivity to the change of glucose before and after the meal was “1”, and this is a reference sensitivity that corresponds to the above-mentioned reference value. The detection sensitivity to the fluctuation (±10%) of water in a single day of the human body was “263” with respect to the reference sensitivity “1”. This result was obtained by adjusting the moisture contained in the artificial skin, calculating the difference in absorbance and determining the ratio of the absorbance difference to the reference value of the detection sensitivity. The detection sensitivity for the variation (±10%) of the thickness of the epidermis layer 30 was “22” with respect to the reference sensitivity “1”. This result was obtained by adjusting the thickness of the artificial skin, calculating the difference in absorbance and determining the ratio of the absorbance difference to the reference value of the detection sensitivity. That is, 22 absorbance fluctuations were observed with respect to the thickness change and 263 absorbance fluctuations were observed with respect to the moisture change when the absorbance fluctuation with respect to before-and-after meal was set to “1”. In particular, the large change in absorbance occurs relative to the moisture change due to the fact that the concentration of glucose in the dermis layer 20 is 0.1% to 1.0%, whereas the concentration of moisture is as high as about 50%. Since moisture absorbs light, moisture is dominant in the dermis layer with respect to changes in absorbance.

### About the Optical Absorption Spectrum

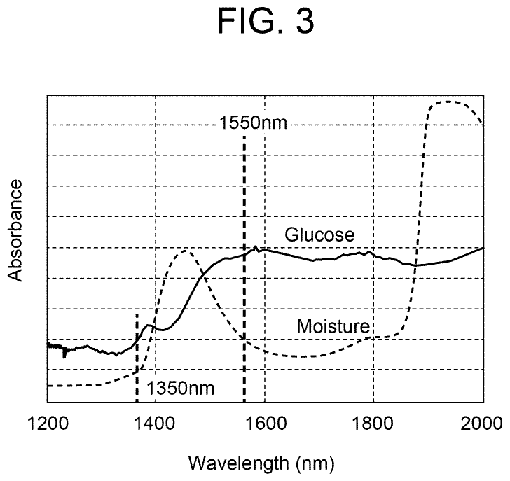

FIG. 3 shows the light absorption spectra of glucose alone and moisture alone. In FIG. 3, the horizontal axis represents wavelength (nm), and the vertical axis represents absorbance. In FIG. 3, a solid line represents a light absorption spectrum of glucose alone, and a broken line represents a light absorption spectrum of moisture alone. FIG. 3 is a graph showing the optical absorption spectra of glucose and moisture. The graph of FIG. 3 is obtained by using a machine that utilizes FTIR (Fourier Transform Infrared Spectroscopy) to measure the absorption spectra of infrared light specific to an object (measurement target).

As shown in FIG. 3, the near-infrared region having a wavelength 1350 nm or less has a low absorbance of glucose and is a wavelength region that is difficult to use as a sensor. In addition, at the wavelength at which the light absorption spectrum of glucose intersects with the light absorption spectrum of moisture, it is not possible to determine whether the change in absorbance is caused by glucose or moisture. If the near-infrared light of the wavelength 1550 nm, with which the absorbance of glucose is relatively high, is used, the difference in absorbance between glucose and moisture increases. However, due to the absorption of light by moisture, the influence of moisture on absorbance is inevitable. For this reason, it is necessary to consider absorption of light by moisture when the near-infrared light is used. In addition, since the above-described “other components” (tolazamide and triglyceride) exhibit absorbances different from those of glucose and moisture for each wavelength, it is also necessary to consider the “other components”.

### Configuration of the Estimating Device

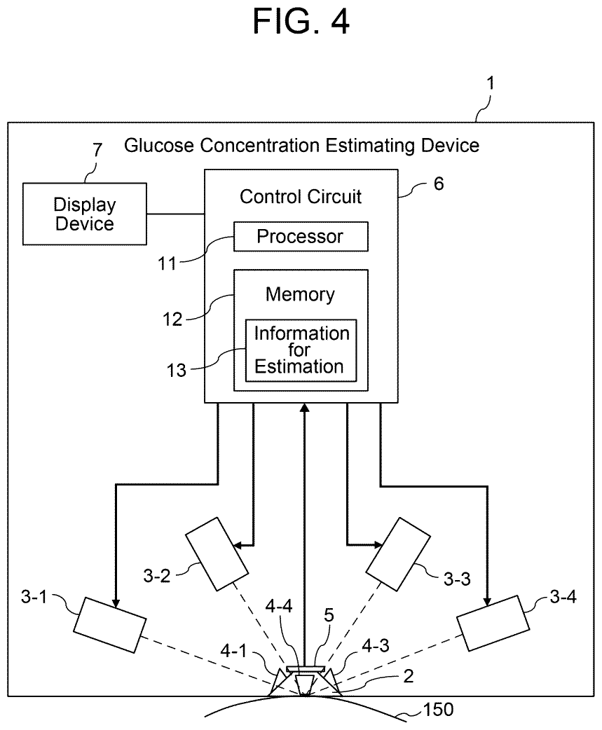

FIG. 4 shows a schematic configuration of the glucose concentration estimating device 1 according to the first embodiment of the present disclosure. The glucose concentration estimating device 1 includes a trapezoidal prism 2, a first light source 3-1, a second light source 3-2, a third light source 3-3, and a fourth light source 3-4. The glucose concentration estimating device 1 also includes a first triangular prism 4-1, a second triangular prism 4-2 (not shown in FIG. 4), a third triangular prism 4-3, a fourth triangular prism 4-4, a light receiving module 5, a control circuit 6, and a display device 7. The light receiving module 5 may have a light receiving element and a circuit.

The first light source 3-1 emits first light including far-infrared light having a predetermined wavelength. The first light is near-infrared light including light of any one wavelength from 1375 nm to 1395 nm wavelength range. The first light source 3-1 is, for example, a laser diode that emits the first light.

The second light source 3-2 emits second light including far-infrared light having a predetermined wavelength. The second light is near-infrared light including light of any one wavelength from 1575 nm to 1595 nm wavelength range. The second light source 3-2 is, for example, a laser diode that emits the second light.

The third light source 3-3 emits third light including far-infrared light having a predetermined wavelength. The third light is near infrared light including light of any one wavelength from 1835 nm to 1855 nm wavelength range. The third light source 3-3 is, for example, a laser diode that emits the third light.

The fourth light source 3-4 emits fourth light including far-infrared light having a predetermined wavelength. The fourth light is near infrared light including light of any one wavelength from 2175 nm to 2255 nm wavelength range. The fourth light source 3-4 is, for example, a laser diode that emits the fourth light.

The above-described “near-infrared light including light of any one wavelength” may be near-infrared light including light of any one wavelength included in the above-mentioned wavelength range. For example, the above-described “near-infrared light including light of any one wavelength” may be light having only one wavelength in the above-mentioned wavelength range, or light having the sole wavelength in the above-mentioned wavelength range as its peak wavelength, together with an unnecessary spectrum (spectrums) that is an unnecessary wavelength component (components). If the light emitted by the light source includes an unnecessary spectrum, a filter that removes the unnecessary spectrum may be used.

Each of the first light source 3-1 to the fourth light source 3-4 is provided with a collimating lens therein so as to be able to emit collimated light. It should be noted that the collimating lenses may be provided outside the first to fourth light sources 3-1 to 3-4, respectively.

The trapezoidal prism 2 has two parallel surfaces of different sizes and four side walls. The four side walls are inclined relative to the two parallel surfaces and connect the two parallel surfaces to each other. The trapezoidal prism 2 is an optical element filled with a medium that transmits predetermined light. The predetermined light is the light used for the measurement, i.e. the first light to the fourth light. The medium that transmits the predetermined light is a light-transmitting material such as glass or transparent plastic.

Hereinafter, a surface having a larger area among the two parallel surfaces of the trapezoidal prism 2 is referred to as a bottom surface, and the other surface, i.e., a surface having a smaller area among the two parallel surfaces of the trapezoidal prism 2, is referred to as a top surface.

When the glucose concentration is estimated, the human body 150 is pressed against the bottom surface of the trapezoidal prism 2. In the illustrated embodiment, the human body 150 is a finger, a wrist, or an arm. It should be noted that the portion of the human body 150 that is pressed against the bottom surface of the trapezoidal prism 2 is not limited to the finger, the wrist, or the arm.

Each of the first triangular prism 4-1 to the fourth triangular prism 4-4 is an optical element that has a triangular prism shape and is filled with a medium that transmits light used for measurement. The refractive index of each of the first triangular prism 4-1 to the fourth triangular prism 4-4 is equal to the refractive index of the trapezoidal prism 2. The medium that transmits the predetermined light is a light-transmitting material such as glass or transparent plastic.

The first light to the fourth light emitted by the first light source 3-1 to the fourth light source 3-4 enter the trapezoidal prism 2 through the first triangular prism 4-1 to the fourth triangular prism 4-4 from the surfaces of the four side walls of the trapezoidal prism 2, respectively. The first light to the fourth light incident on the trapezoidal prism 2 reach the bottom surface of the trapezoidal prism 2 and enter the human body 150 pressed against the bottom surface of the trapezoidal prism 2. On the other hand, the reflected light of the first light to the fourth light returning from the human body 150 is incident on the bottom surface of the trapezoidal prism 2, and is condensed on the top surface of the trapezoidal prism 2 directly without experiencing multiple reflection in the trapezoidal prism 2, or after experiencing the multiple reflection in the trapezoidal prism 2.

The light receiving module 5 is disposed on the top surface of the trapezoidal prism 2. The light receiving module 5 may be any suitable module as long as it can detect the first light to the fourth light. For example, a module including a light receiving element using GaAs may be employed as the light receiving module 5.

Although not shown, the light receiving module 5 has a configuration in which the light receiving element and a circuit are sealed with resin. For example, the light receiving element is a photodiode, and the circuit is an IC (integrated circuit) in which a driving circuit and an arithmetic circuit of the light receiving element are configured by a single chip. In the light receiving module 5, a transparent plate such as a glass plate is provided on a light receiving surface of the light receiving element such that the surface of the glass plate is exposed, and the glass plate and the light receiving element are integrally molded with resin.

When the first light to the fourth light are incident on the light receiving module 5 through the top surface of the trapezoidal prism 2, the light receiving module 5 generates an electric signal corresponding to the intensity of the incident light. The electric signal generated from the light receiving module 5 is introduced to the control circuit 6.

The control circuit 6 drives the first light source 3-1, the second light source 3-2, the third light source 3-3, the fourth light source 3-4, and the light receiving module 5 to estimate the concentration of glucose in the blood of the living body 150 based on the intensity of the first light to the fourth light detected by the light receiving module 5. As a configuration for this purpose, the control circuit 6 includes a processor 11 and a memory 12.

The memory 12 stores, in advance, estimation information (information used in an estimating process) 13 for estimating the concentration of glucose in the blood from the output signal of the light receiving module 5. In addition, various control programs necessary for estimating the glucose concentration are stored in the memory 12 in advance.

The estimation information 13 includes, for example, a learned model in which the relationship between the information of the reflected light of the first light to the fourth light incident on the human body 150 and the glucose concentration is learned. The estimation information 13 also includes an information table generated by using the learned model. The information table indicates the relationship between the information of the reflected light of the first light to the fourth light and the glucose concentration. The learned model is, for example, a model that accepts, as its input, a detection value (i.e., a voltage value of an output signal of the light receiving module 5 that has received the reflected light of the first light, the reflected light of the second light, the reflected light of the third light and the reflected light of the fourth light), and generates, as its output, a glucose concentration. That is, the detection value is the information of the reflected light. It should be noted that the learned model may be configured to generate other values that can be converted into the glucose concentration without outputting the glucose concentration itself.

The estimation information 13 may include both the learned model and the information table, or may include only one of them.

A learning method of the learned model may be a method of performing learning that uses information of reflected light of the first to fourth light with respect to a plurality of human bodies 150 (a plurality of test subjects) and actual measurement values of the glucose concentration. In this case, for example, the blood of each of the test subjects is collected to obtain the actual measurement value of the glucose concentration.

Alternatively, the learning method may use artificial skin called a phantom. In this method, the concentration of glucose, moisture, and a third component are set for the dermis layer of the artificial skin, and learning is performed for a plurality of different combinations of the glucose, the moisture and the third component while changing the concentrations of the glucose, the moisture and the third component. The third component is, for example, nanocellulose or the like. The third component is a component that can replace the above-mentioned “other components” such as tolazamide and triglyceride. If the learning method uses the artificial skin, a learned model for an unspecified number of persons (human bodies) is generated.

The learning algorithm may be, for example, a linear regression analysis, MARS (Multivariate Adaptive Regression Splines), support vector regression (SVR), regression tree, model tree, gene program, binary classification, logistic regression, k-neighborhood method, support vector machine, decision tree, random forest, or neural network.

In the first embodiment, a learned model is generated by using deep learning, which is one of learning methods using a neural network.

The estimation information 13 may include, for example, an information table indicating relationship among information of the reflected light of the first to fourth light, attribute information of the test subjects and the glucose concentration when learning is performed using the attribute information of the test subjects.

The attribute information includes, for example, information such as age, sex, height, and weight of each of the test subjects.

The processor 11 causes the first light source 3-1, the second light source 3-2, the third light source 3-3, and the fourth light source 3-4 to emit light simultaneously or at different timings.

If the processor 11 triggers simultaneous light emission of the four light sources 3-1, 3-2, 3-3 and 3-4, the processor 11 acquires a signal indicating the intensity of the reflected light of the mixed light of the first to fourth light, which is generated from the light receiving module 5.

On the other hand, if the processor 11 causes the first light source 3-1, the second light source 3-2, the third light source 3-3, and the fourth light source 3-4 to emit light at different timings, the processor 11 acquires a signal indicating the intensity of the reflected light of the first light, a signal indicating the intensity of the reflected light of the second light, a signal indicating the intensity of the reflected light of the third light, and a signal indicating the intensity of the reflected light of the fourth light, which are respectively generated from the light receiving module 5.

The processor 11 also acquires the attribute information of one test subject to be estimated if the estimation information 13 includes an information table associated with the attribute information of the test subject. Then, the processor 11 estimates the concentration of glucose in the blood of this test subject based on the acquired signals, the attribute information, and the estimation information 13.

On the other hand, when the estimation information 13 includes a learned model using the artificial skin or an information table generated using the learned model, the processor 11 estimates the concentration of glucose in the blood of the test subject based on the acquired signals and the estimation information 13.

The processor 11 may be constituted by, for example, a CPU (Central Processing Unit), or may be realized by hardware (circuitry) using, for example, an FPGA (Field-Programmable Gate Array) or an ASIC (Application Specific Integrated Circuit).

The control circuit 6 may include an amplifier circuit for amplifying a signal from the light receiving module 5, an analog-to-digital conversion circuit for converting a signal into a digital value, and the like.

The control circuit 6 sends the estimated concentration of glucose in the blood to the display device 7 as an estimation result.

The displaying device 7 is, for example, an LCD (Liquid Crystal Display) or an OELD (Organic Electroluminescent Display). The display device 7 displays, on its screen, the concentration of glucose in the blood, which is received from the control circuit 6, in a manner visible to a user of the glucose concentration estimating device 1. The display device 7 may display the glucose concentration as numerical information, or may display the glucose concentration by a graph, a bar, or the like.

It should be noted that the glucose concentration estimating device 1 does not necessarily have to include the display device 7. The glucose concentration estimating device 1 may include a speaker instead of the display device 7 such that the speaker generates, in the form of audio information, the estimated concentration of glucose in the blood.

Further, the glucose concentration estimating device 1 may include a wired or wireless interface that can connect to an external device such as a personal computer or a smartphone, and the control circuit 6 may send the estimated concentration of glucose in the blood to the external device via the interface.

### Configuration of the Trapezoidal Prism 2 and the Arrangement of the Components Around the Trapezoidal Prism 2

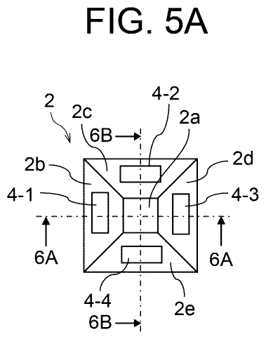

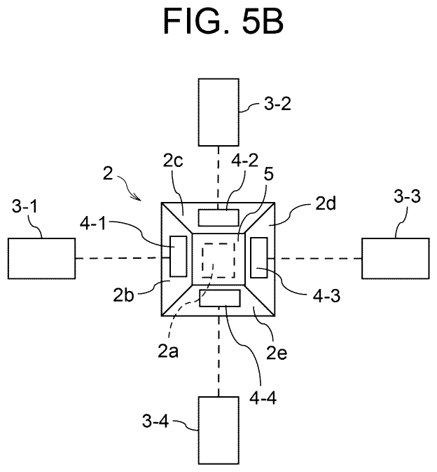

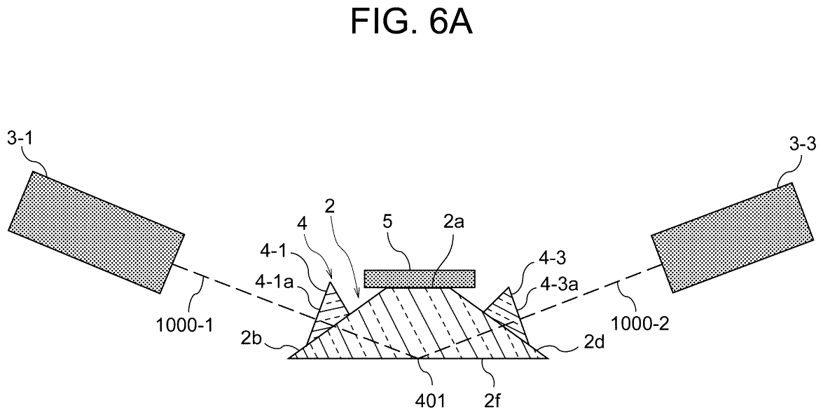

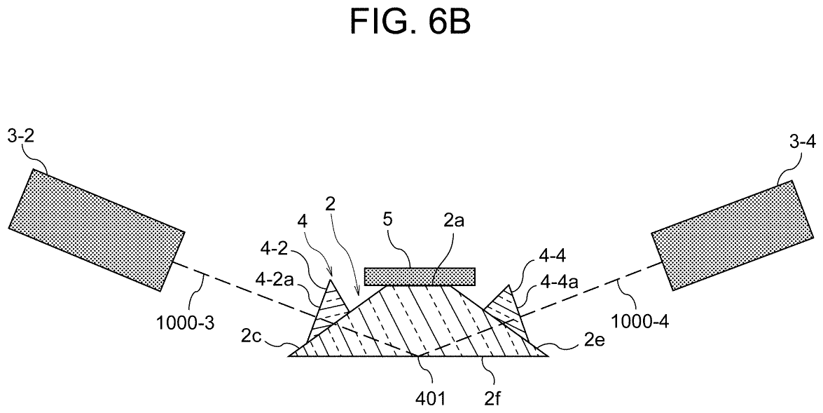

FIG. 5A is a top view of the trapezoidal prism 2 and the first triangular prism 4-1 to the fourth triangular prism 4-4 of the first embodiment. FIG. 5B shows the arrangement of the trapezoidal prism 2, the first triangular prism 4-1 to the fourth triangular prism 4-4, and the first light source 3-1 to the fourth light source 3-4. FIG. 6A shows the incidence of the first light to the first triangular prism 4-1 and the incidence of the third light to the third triangular prism 4-3. FIG. 6B shows the incidence of the second light to the second triangular prism 4-2 and the incidence of the fourth light to the fourth triangular prism 4-4.

The cross-sectional views of the trapezoidal prism 2 and the first triangular prism 4-1 and the third triangular prism 4-3 in FIG. 6A are taken along the 6A-6A line in FIG. 5A. The cross-sectional views of the trapezoidal prism 2 and the second triangular prism 4-2 and the fourth triangular prism 4-4 in FIG. 6B are taken along the 6B-6B line in FIG. 5A.

In FIG. 6A, reference numeral 1000-1 denotes an optical path of the first light emitted from the first light source 3-1, and reference numeral 1000-3 denotes an optical path of the third light emitted from the third light source 3-3. In FIG. 6B, reference numeral 1000-2 denotes an optical path of the second light emitted from the second light source 3-2, and reference numeral 1000-4 denotes an optical path of the fourth light emitted from the fourth light source 3-4.

As shown in FIGS. 5A, 5B, 6A, and 6B, the surface 2a of the trapezoidal prism 2 is a top surface on which the light-receiving module 5 is disposed. The surfaces 2b, 2c, 2d and 2e of the trapezoidal prism 2 are side walls, and the surface 2f is a bottom surface pressed against the human body 150. Hereinafter, the surface 2a is referred to as a top surface 2a, and the surface 2f is referred to as a bottom surface 2f. The surfaces 2b, 2c, 2d and 2e are referred to as sidewall 2b, 2c, 2d and 2e, respectively. The first light enters the sidewall 2b, the second light enters the sidewall 2c, the third light enters the sidewall 2d, and the fourth light enters the sidewall 2e.

The intensity of the light that is reflected in the dermis layer and returns back is strongest at the irradiation position of the light, and decreases with increasing distance from the irradiation position of the light. Therefore, the positions and orientations of the first light source 3-1 to the fourth light source 3-4 are determined so that the first light to the fourth light are emitted to the human body 150 from the position 401 of the center of the area of the bottom surface 2f, which faces the top surface 2a. When the bottom surface 2f of the trapezoidal prism 2 is pressed against the human body 150, the light emitted from the position 401 into the human body 150 returns from the position 401 with strong intensity. Since the light receiving module 5 is located directly in front of the position 401 (the light receiving module 5 faces the position 401 with a minimum distance) where the light strongly returns, the light receiving module 5 can receive a large amount of the return light from the human body 150.

On the side wall 2b where the first light enters, the first triangular prism 4-1 is arranged in order to suppress the optical loss due to the reflection of the first light. On the side wall 2d where the third light enters, the third triangular prism 4-3 is arranged in order to suppress the optical loss due to the reflection of the third light. On the side wall 2c where the second light enters, the second triangular prism 4-2 is arranged to suppress the optical loss due to the reflection of the second light. On the side wall 2e where the fourth light enters, the fourth triangular prism 4-4 is arranged to suppress the optical loss due to the reflection of the fourth light.

Specifically, the first light enters the wall surface 4-1a of the first triangular prism 4-1 perpendicularly, the second light enters the wall surface 4-2a of the second triangular prism 4-2 perpendicularly, the third light enters the wall surface 4-3a of the third triangular prism 4-3 perpendicularly, the fourth light enters the wall surface 4-4a of the fourth triangular prism 4-4 perpendicularly. The refractive index of each of the first triangular prism 4-1 to the fourth triangular prism 4-4 is equal to the refractive index of the trapezoidal prism 2.

### Suitable Wavelengths of the First Light to the Fourth Light

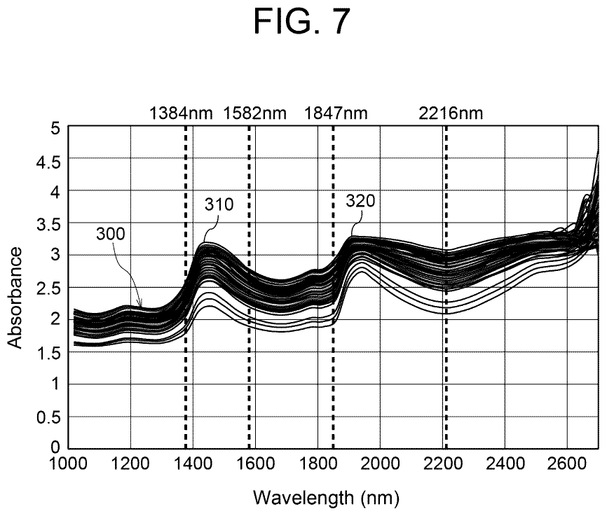

FIG. 7 is a graph showing the relationship between wavelength and absorbance, which is obtained by using artificial skin that contains glucose, moisture, and nanocellulose under mixed conditions. Curves in FIG. 7 show the absorbances when concentrations of the glucose, moisture and third component in the artificial skin are altered. In FIG. 7, the horizontal axis represents the wavelength (nm) and the vertical axis represents the absorbance.

In FIG. 7, a group of waveforms (curves) 300 include a plurality of types of waveforms corresponding to different combinations of concentrations of glucose, moisture, and third component(s) in the artificial skin.

The waveform group 300 has two absorbance peaks, i.e., a first peak 310 and a second peak 320. In the wavelength regions in the vicinity of the first peak 310 and the second peak 320, the variation of the absorbance is too large, and therefore the estimation error becomes large when the wavelengths in these regions are used. On the other hand, the variation of the absorbance is relatively small at the wavelengths in the wavelength regions of the two tail portions (right and left lower adjacent portions) of the first peak 310 and the two tail portions of the second peak 320. Thus, the absorbance is stable in these four wavelength regions.

Therefore, in the first embodiment, four wavelengths are adopted as the wavelengths of the first to fourth light. Specifically, one wavelength selected from the left tail portion of the first peak 310, one wavelength selected from the right tail portion of the first peak 310, one wavelength selected from the left tail portion of the second peak 320, and one wavelength selected from the right tail portion of the second peak 320 are used for the wavelengths of the first to fourth light, respectively.

In the embodiment shown in FIG. 7, as indicated by four broken lines in the drawing, 1384 nm is selected as the wavelength of the first light, 1582 nm is selected as the wavelength of the second light, 1847 nm is selected as the wavelength of the third light, and 2216 nm is selected as the wavelength of the fourth light.

Although this embodiment describes an example in which the above-mentioned four wavelengths are selected, the wavelength of the first light can be selected from the wavelength range of 1375 nm to 1395 nm, and the wavelength of the second light can be selected from the wavelength range of 1575 nm to 1595 nm. The wavelength of the third light can be selected from a wavelength range of 1835 nm to 1855 nm, and the wavelength of the fourth light can be selected from a wavelength range of 2175 nm to 2255 nm.

### Example of the Learning Method

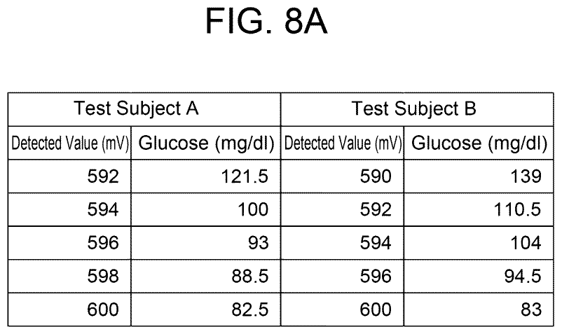

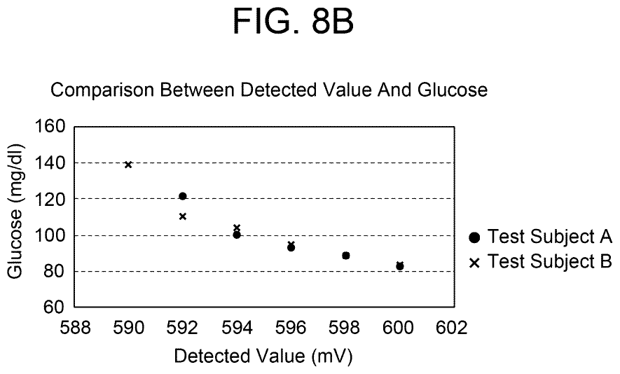

FIG. 8A is a table that shows the relationship between the detected values (mV) of the reflected lights of the test subjects A and B and the measured values of the glucose concentration (mg/dl). FIG. 8B graphically depicts the table of FIG. 8A.

In FIGS. 8A and 8B, the first light to the fourth light are simultaneously emitted onto the skin for each test subject A, B, and the detected reflected light and the measured glucose concentration are learned many times. That is, a learned model is generated for each test subject A, B.

In this embodiment, the detected value is a voltage value of the output signal indicating the intensity of the reflected light of the light receiving module 5, and the unit is mV.

FIG. 8A shows the two test subjects A and B, but the test subjects may not be particular individuals. If the test subjects are not particular individuals, it is desirable that a learned model is made by learning about a large number of test subjects having different attributes (properties) such as different ages, different sexes, different weights, and different heights.

FIG. 8B indicates that even if two detected values are close to each other, the blood glucose level of one person differs from that of another person depending on the individual differences.

It should be noted that although the four lights (first to fourth light) are simultaneously emitted and one detection value is obtained in the above-described embodiment, the four lights may be sequentially emitted at different timings such that four detection values are obtained and four measured values of the glucose concentration are obtained. Then, a relationship between the four detection values and the four measured values of the glucose concentration may be learned.

Still another learning method may be employed. Although not shown, the concentration of moisture, the concentration of glucose, and the concentration of the third component may be changed with respect to the artificial skin, and the detection values and the measured values may be learned many times. In this case, a learned model for an unspecified number of people is generated. Also in this case, the four lights (first to fourth light) may be emitted simultaneously to the artificial skin such that the relationship between one detection value of the reflected lights and one measured value of the glucose concentration may be learned, or the four lights may be emitted to the artificial skin at different timings such that the relationship between four detection values and four measured values may be learned.

### Glucose Concentration Estimating Process

A glucose concentration estimating process executed by the control circuit 6 will now be described.

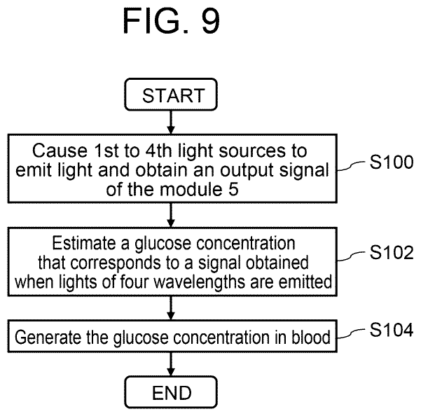

FIG. 9 is a flowchart illustrating a glucose concentration estimating process according to the first embodiment.

The processor 11 of the control circuit 6 activates a control program stored in a predetermined area of the memory, and executes the glucose concentration estimating process illustrated in the flowchart of FIG. 9 in accordance with the control program.

When the glucose concentration estimating process is executed in the processor 11, first, the process proceeds to Step S100 as shown in FIG. 9.

In Step S100, the first light source 3-1 to the fourth light source 3-4 are caused to emit light such that the human body 150 of one test subject to be estimated is irradiated with the first to fourth light. The light receiving module 5 receives the reflected light of the first to fourth light, which returns from the human body 150, and generates an output signal. Then, the processor 11 (or the control circuit 6) obtains the output signal of the light receiving module 5. Thereafter, the estimating process proceeds to Step S102.

In Step S102, the processor 11 uses the estimation information 13 to estimate the glucose concentration. The estimation information 13 includes a learned model. The learned model to be used is decided, depending on whether the first to fourth lights are emitted simultaneously or at different timings. If the four lights (first to fourth lights) are simultaneously emitted to the human body, the learned model provides the relationship between the light detection value and the measured value of the glucose concentration. It should be noted that the estimation information 13 may include an information table generated from this learned model. The first light source 3-1 to the fourth light source 3-4 are caused to emit light simultaneously such that the four lights (first to fourth lights) are simultaneously emitted to the human body, In this instance, the output signal of the light receiving module 5 in Step S100 represents a value acquired when the light receiving module 5 receives the reflected light from the human skin upon simultaneous emission of the four lights.

On the other hand, if the four lights (first to fourth lights) are emitted to the human body at different timings, the estimation information includes another learned model that provides the relationship between the light detection values and the measured values of the glucose concentration value when the first light source 3-1 to the fourth light source 3-4 emit light individually at different timings. It should be noted that the estimation information 13 may include an information table generated from this learned model. The first light source 3-1 to the fourth light source 3-4 are caused to emit light at different timings such that the four lights (first to fourth lights) are emitted to the human body at different timings. In this instance, the light receiving module 5 receives the four reflected lights from the human skin, and therefore the light receiving module 5 generates four output signals in the order of the light irradiation (emission). That is, the four output signals corresponding to the four reflected lights are acquired.

In Step S102, the estimation information 13 stored in the memory 12 is used to estimate the glucose concentration of one test subject that corresponds to the detected value indicated by the acquired signal for the light emission of the four wavelengths. Thereafter, the estimating process proceeds to Step S104.

Here, it is assumed that the estimation information 13 includes a plurality of information tables. Each of the information tables indicates the relationship between the attribute information of the test subject, the detection value, and the glucose concentration. Each of the information tables is generated from the learned model in which the relationship between the detected value of the reflected light and the measured value of the glucose concentration with respect to the irradiation (emission) of the first light to the fourth light is learned for each test subject. To apply the estimating process of FIG. 9 to a particular test subject, one information table should be selected from a plurality of information tables in the estimation information 13. To this end, the attribute information of a particular test subject is acquired, and one information table corresponding to the attribute information identical to or closest to the acquired attribute information is selected from the information tables. Then, the glucose concentration of this test subject is estimated by acquiring, from the selected information table, the glucose concentration corresponding to the detection value that is the same as or closest to the detection value of this test subject.

When the estimation information 13 includes only the learned model and does not include the information table, the learned model corresponding to the attribute information that is the same as or closest to the acquired attribute information of the test subject is selected. That is, the attribute information and the learned model are associated with each other in advance. Then, a detected value corresponding to the output signal acquired in Step S100 is entered to the selected learned model, and an output of the learned model is taken as an estimated value of the glucose concentration. When the output value of the learned model does not directly indicate the glucose concentration, the value converted into the glucose concentration is used as the estimated value.

On the other hand, if the estimation information 13 includes an information table generated from the learned model, which has learned the relationship between the detected value of the reflected light and the glucose concentration when the artificial skin is irradiated with the first to fourth light, this information table indicates a relationship between the detected value and the glucose concentration, In this instance, the glucose concentration of the test subject is estimated by acquiring, from the information table, the glucose concentration corresponding to the detection value that is the same as or closest to the detection value of the test subject.

When the estimation information 13 includes only the learned model and does not include the information table, a detection value corresponding to the acquired output signal is entered to the learned model, and an output of the learned model is taken as an estimated value of the glucose concentration.

In Step S104, the glucose concentration in the blood estimated by Step S102 is displayed on the display device 7. Then, the estimating process ends.

### Estimation Accuracy When Four Wavelengths are Used

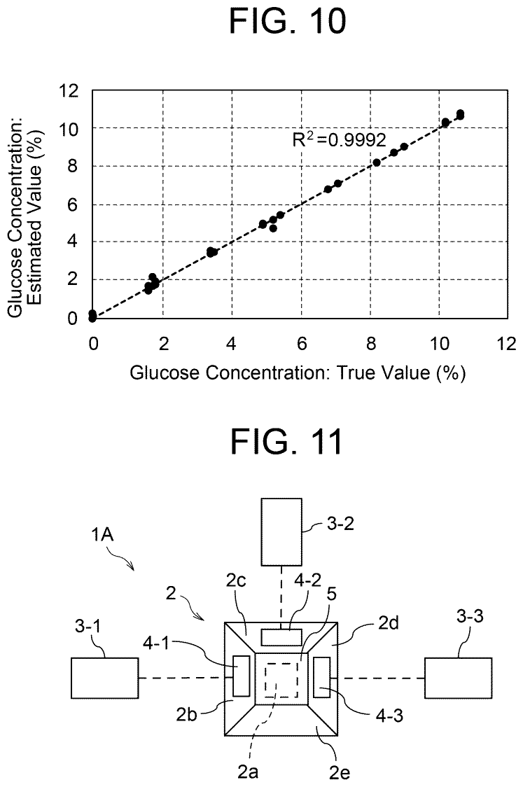

FIG. 10 shows a relationship between a true value and an estimated value in a case where a detection value of reflected light returning upon emitting light of four wavelengths is used. In FIG. 10, the horizontal axis represents a true value of the glucose concentration, and the vertical axis represents an estimated value of the glucose concentration. R² in FIG. 10 is a determination factor, and indicates that the closer to 1, the more accurate the estimation is performed. The dashed line in FIG. 10 is an approximate straight line.

It can be understood from FIG. 10 that the estimated value and the true value substantially coincide with each other as a result of learning based on the detected value when “1384 nm”, “1582 nm”, “1847 nm”, and “2216 nm” are used as the four wavelengths. The determination coefficient becomes “0.9992”, and it is understood that highly accurate estimation has been performed.

The estimation result shown in FIG. 10 is an estimation result in a case where learning is performed using the artificial skin.

As described above, learning was performed by changing the concentrations of the three components contained in the artificial skin, namely, glucose, water, and the third component. In a second embodiment of the present disclosure, which will be described later, the light sources having three wavelengths are used, and the estimation accuracy is also improved as compared to a conventional device but the estimation accuracy of the first embodiment is significantly higher than the second embodiment. The inventor has inferred a reason why the estimation accuracy is significantly higher in the first embodiment than the estimation accuracy in the second embodiment. When the light sources of three wavelengths were used, the determination coefficient was “0.7996”. This is presumably because there was a measurement error of the sensor such as a deviation from an intended location of the sensor upon attaching (pressing) the sensor to the skin to measure the true value. On the other hand, in the first embodiment, even if there is a measurement error of the sensor, the determination coefficient becomes “0.9992” because of use of the four wavelengths. Thus, when the measurement error is considered, it is presumed that the estimation accuracy is remarkably improved when the four wavelengths are used. It is presumed that such a remarkable improvement in estimation accuracy was obtained in the first embodiment by using the light sources with the four wavelengths or the four wavelength ranges including the four wavelengths.

### Advantages of the First Embodiment

As described above, the glucose concentration estimating device 1 of the first embodiment includes the first light source 3-1 for irradiating the human body 150 with the first light containing the light of any one wavelength in the wavelength range of 1375 nm to 1395 nm, the second light source 3-2 for irradiating the human body 150 with the second light containing the light of any one wavelength in the wavelength range of 1575 nm to 1595 nm, the third light source 3-3 for irradiating the human body 150 with the third light containing the light of any one wavelength in the wavelength range of 1835 nm to 1855 nm, the fourth light source 3-4 for irradiating the human body with the light of any one wavelength in the wavelength range of 2175 nm from 2255 nm, the light receiving module 5 having a light receiving element for receiving the light returning from the human body 150 upon irradiating the huma body 150 with the first to fourth light, and the control circuit 6 for estimating the glucose concentration based on the output of the light receiving module 5.

With this configuration, the glucose concentration can be estimated on the basis of information on the reflected light upon emitting the light at the four wavelengths in the four wavelength ranges in which the difference in absorbance between the glucose and the moisture contained in the dermis layer is relatively large and the absorbance is stable. As a result, the glucose concentration corresponding to the change in the absorbance due to moisture can be estimated, and thus the glucose concentration can be estimated with high accuracy. In addition, a relatively inexpensive laser diode can be adopted as each of the first light source 3-1 to the fourth light source 3-4, and each of the first light to the fourth light has a wavelength in a wavelength range to which a special (expensive) optical material is not required. Thus, the device 1 for estimating the glucose can be manufactured at a lower cost than a conventional device.

Further, the glucose concentration estimating device 1 of the first embodiment includes the memory 12 to store the estimation information 13, and the estimation information 13 includes the learned model obtained by learning in advance the relationship between the information of the reflected light and the glucose concentration when the first light to the fourth light are incident on the human body 150. The glucose concentration estimating device 1 also includes the control circuit 6 to estimate the glucose concentration corresponding to the output of the light receiving module 5 using the estimation information 13 stored in the memory 12.

With this configuration, the glucose concentration can be estimated based on the estimation information 13 including the learned model in which the relationship between the information of the reflected light and the glucose concentration is learned when the first light to the fourth light are emitted to the human body 150. Accordingly, it is possible to estimate, with high accuracy, the glucose concentration even if the absorbance changes due to moisture.

Further, the learned model used by the glucose concentration estimating device 1 of the first embodiment is a model in which learning is conducted with respect to a combination of the glucose, the moisture, and the third component (this corresponds to other components in the dermis layer than the glucose and the moisture) set for the artificial skin while altering the concentrations of the glucose, the moisture and the third component respectively.

With this configuration, it is not necessary to measure a large number of people as test subjects, and it is possible to easily perform learning by setting a combination of desired concentrations. As a result, a learned model that can cope with an unspecified number of people can be easily generated.

Further, the estimation information 13 of the glucose concentration estimating device 1 of the first embodiment includes the information table that indicates the relationship between the information of the reflected light and the glucose concentration. The information table is generated by using the learned model that is obtained as the relationship between the information of the reflected light and the glucose concentration upon emitting the first to fourth light to the human body 150 is learned in advance. The control circuit 6 estimates the glucose concentration corresponding to the output of the light receiving module 5 based on the estimation information 13 stored in the memory 12.

With this configuration, the glucose concentration can be estimated by selecting the glucose concentration corresponding to the output of the light receiving module 5 from the information table. Thus, the estimation can be performed by a simple process as compared with an arithmetic processing by the learned model.

The control circuit 6 of the glucose concentration estimating device 1 of the first embodiment acquires the attribution information about a test subject's attribute, and the learned model is a model in which the relationship between the reflected light information and the glucose concentration upon emitting the first to fourth light to the skin of the test subject is learned, based on the information on the reflected light of the first to fourth light emitted to the skin of the test subject and the actual measurement of the glucose concentration of the test subject, for each of a plurality of test subjects. The estimation information 13 has the information table generated by using the learned model, and the information table indicates the relationship among the attribution information of the test subject, the reflected light information of the test subject corresponding to the attribute information, and the glucose concentration of the test subject corresponding to the attribution information. The control circuit 6 uses the information table to estimate the glucose concentration of the test subject concerned, on the basis of the attribute information of the test subject concerned, and the output of the light receiving module 5 for the test subject concerned.

With this configuration, the glucose concentration of the test subject concerned can be estimated by using an information table generated from a learned model generated for another test subject having the same or a similar attribute to the test subject concerned. This makes it possible to easily and accurately estimate the glucose concentration(s) for two or more test subjects having the same attribute or a similar attribute.

### Relationship Between the First Embodiment and the Claims

The control circuitry 6 and Step S102 in the first embodiment correspond to an estimating unit and an attribution information acquiring unit in the claims. The human body 150 corresponds to a living body in the claims. The memory 12 corresponds to a storage unit in the claims.

### Second Embodiment

A second embodiment of the present disclosure will now be described with reference to FIG. 11 to FIG. 13. Same or similar reference numerals are used in the first and second embodiment to denote same or similar elements.

### Configuration

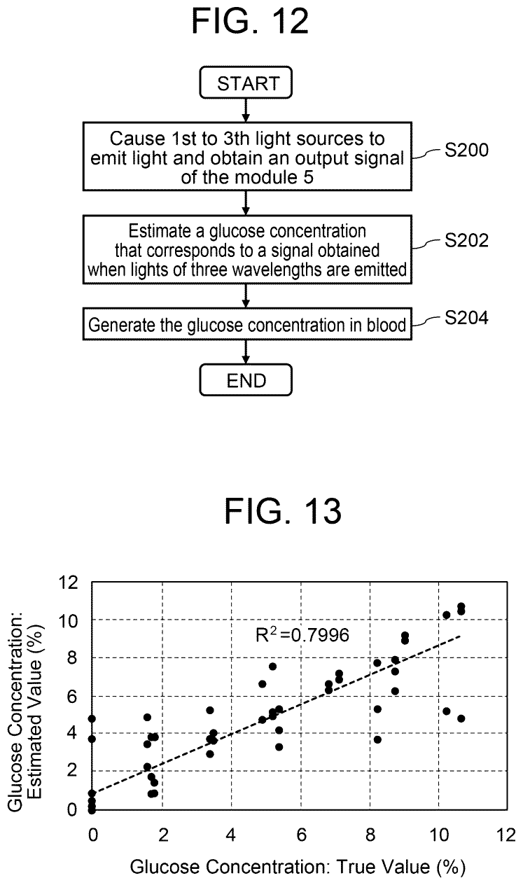

FIG. 11 illustrates a part of a schematic configuration of a glucose concentration estimating device 1A according to the second embodiment of this disclosure. FIG. 12 is a flowchart illustrating a glucose concentration estimating process according to the second embodiment. FIG. 13 is a diagram illustrating a relationship between a true value and an estimated value when a detection value obtained with three wavelengths is used.

The second embodiment is different from the first embodiment in that the first to third light of the three wavelengths used in the first embodiment is incident on the human body 150 in the second embodiment (i.e., the fourth light is not used), and the glucose concentration is estimated based on the detected value of the reflected light of the first to third light in the second embodiment.

As shown in FIG. 11, the glucose concentration estimating device 1A of the second embodiment has a configuration in which the fourth light source 3-4 and the fourth triangular prism 4-4 are removed from the glucose concentration estimating device 1 of the first embodiment.

The first light is incident on the sidewall 2b through the first triangular prism 4-1, the second light is incident on the sidewall 2c through the second triangular prism 4-2, and the third light is incident on the sidewall 2d through the third triangular prism 4-3.

As in the first embodiment, “1384 nm” is adopted as the wavelength of the first light emitted by the first light source 3-1, “1582 nm” is adopted as the wavelength of the second light emitted by the second light source 3-2, and “1847 nm” is adopted as the wavelength of the third light emitted by the third light source 3-3.

The estimation information 13 used in the second embodiment includes, for example, a learned model in which the relationship between the detected value of the reflected light of the three lights (the first light, the second light and the third light) incident on the human body 150 and the glucose concentration is learned, and an information table generated using the learned model such that the information table indicates the relationship between the reflected light of the first to third light and the glucose concentration. The learned model is, for example, a model in which a detection value (i.e., a voltage value of an output signal of the light receiving module 5 that has received the reflected light of the first to third light) is used as an input, and a glucose concentration is used as an output.

Similar to the first embodiment, the estimation information 13 used in the second embodiment may include both the learned model and the information table, or may include only one of them.

The learning method of the learning model is the same as that of the first embodiment except that the detection value of the reflected light with respect to the first to third light is used.

### Glucose Concentration Estimating Process

Next, a glucose concentration estimating process performed by the control circuit 6 according to the second embodiment will be described.

FIG. 12 is a flowchart illustrating a glucose concentration estimating process according to the second embodiment.

The processor 11 of the control circuit 6 activates a control program stored in a predetermined area of the memory 12, and executes the glucose concentration estimation process illustrated in the flowchart of FIG. 12 in accordance with the control program.

When the glucose concentration estimating process is executed in the processor 11, first, the estimating process proceeds to Step S200, as shown in FIG. 12.

In Step S200, the first light source 3-1 to the third light source 3-3 are caused to emit light, the human body 150 of one test subject to be estimated is irradiated with the first light to the third light, and the output signal of the light receiving module 5 that has received the reflected light of the first light to the third light returning from the human body 150 is acquired. Thereafter, the estimating process proceeds to Step S202.

As in the first embodiment, the first light source 3-1 to the third light source 3-3 are caused to emit light at the same time or at different timings individually, depending on the contents of the estimation information 13. Then, an output signal of the light receiving module 5 for each reflected light is acquired.

In Step S202, the estimation information 13 stored in the memory 12 is used to estimate the glucose concentration of the test subject that corresponds to the detected value indicated by the acquired signal (output signal of the light receiving module 5) for the light emission of the three wavelengths. Thereafter, the estimating process proceeds to Step S204.

The second embodiment is different from the first embodiment in that in the second embodiment, the learned model learns the relationship between the detected value of the reflected light for each test subject and the measured value of the glucose concentration with respect to the irradiation of the first light to the third light, and the information table is generated using the learned model. Other processing in the glucose concentration estimating process is the same as the glucose concentration estimating process of the first embodiment.

In Step S204, the glucose concentration in the blood estimated by Step S202 is displayed on the display device 7. Thereafter, the series of processing ends.

Estimation Accuracy when Three Wavelengths are used

FIG. 13 shows a relationship between a true value and an estimated value when a detection value of reflected light of the three lights having the three wavelengths is used. In FIG. 13, the horizontal axis represents a true value of the glucose concentration, and the vertical axis represents an estimated value of the glucose concentration. R² in FIG. 13 is a determination factor, and indicates that the closer to 1, the more accurate the estimation is performed. The broken line in FIG. 13 is an approximate straight line.

As shown in FIG. 13, the relationship between the true values and the estimated values obtained with the three wavelengths are learned based on the detected values with the three wavelengths of “1384 nm”, “1582 nm”, and “1847 nm”. It is understood from FIG. 13 that the estimated values and the true values are more inconsistent with each other as compared with the first embodiment in which the learning is conducted with the four wavelengths. It should be noted, however, that even in the second embodiment, the determination coefficient becomes “0.79962”, and it can be said that the estimation with relatively high accuracy can be performed.

Since the estimation accuracy is higher for the four wavelengths (first embodiment) than for the three wavelengths (second embodiment), it can be predicted that the estimation accuracy can be improved by increasing the number of the wavelengths used in the learning.

The estimation result shown in FIG. 13 is an estimation result obtained when learning is performed using the artificial skin.

### Advantages of the Second Embodiment

As described above, the glucose concentration estimating device 1A of the second embodiment includes the first light source 3-1 for irradiating the human body 150 with the first light including the light of any one wavelength in the wavelength range of 1375 nm to 1395 nm, the second light source 3-2 for irradiating the human body 150 with the second light including the light of any one wavelength in the wavelength range of 1575 nm to 1595 nm, the third light source 3-3 for irradiating the human body 150 with the third light including the light of any one wavelength in the wavelength range of 1835 nm from 1855 nm, the light receiving module 5 having the light receiving element for receiving the light returning upon irradiating the human body 150 with the first light to the third light, and the control circuit 6 for estimating the glucose concentration based on the output of the light receiving module 5.

With this configuration, the glucose concentration can be estimated on the basis of the information on the reflected light with respect to the irradiation of light in the three wavelength ranges in which the difference in absorbance between glucose and moisture contained in the dermis layer is relatively large and the absorbance is stable. As a result, the glucose concentration that can cope with (can change appropriately with) the change in the absorbance due to moisture can be estimated, and thus the glucose concentration can be estimated with high accuracy. In addition, a relatively inexpensive laser diode can be adopted as the first light source 3-1 to the third light source 3-3, and the first light to the third light are the light in the three wavelength ranges, respectively, which can be emitted without a special (expensive) optical material. Therefore, the device 1A for estimating the glucose concentration can be manufactured at a lower cost than a conventional device. Further, as compared with the first embodiment in which the four lights having the four wavelengths respectively are used, the second embodiment can reduce the number of the light sources and the triangular prisms. Thus, it is possible to manufacture the glucose concentration estimating device 1A at a lower cost.

The glucose concentration estimating device 1A according to the second embodiment includes the memory 12 that stores the estimation information 13, and the estimation information 13 includes the learned model obtained by learning in advance the relationship between the information of the reflected light and the glucose concentration when the first light to the third light are incident on the human body 150. The control circuitry 6 estimates the glucose concentration based on the output of the light receiving module 5 by using the estimation information 13 stored in the memory 12.

With this configuration, the glucose concentration can be estimated based on the estimation information 13 that includes the learned model in which the relationship between the information of the reflected light and the glucose concentration with respect to the irradiation (emission) of the first light to the third light to the human body 150 is learned. Accordingly, it is possible to estimate, with higher accuracy, the glucose concentration that appropriately reflects the change in absorbance due to moisture.

In the glucose concentration estimating device 1A according to the second embodiment, the learned model is a model in which a combination of the glucose, the moisture, and the third component (i.e., other components than the glucose and the moisture) in the dermis layer set for the artificial skin is learned many times while altering the concentrations of the glucose, the moisture and the third component in the respective combinations.

With this configuration, it is not necessary to measure a large number of people as test subjects, and it is possible to easily perform learning by setting a combination of desired concentrations. As a result, a learned model that can be used for an unspecified number of people can be easily generated.

In the second embodiment, the estimation information 13 includes an information table that indicates the relationship between the information of the reflected light and the glucose concentration. The information table is generated by using a learned model in which the relationship between the information of the reflection light and the glucose density is learned in advance upon emitting the first to third lights to the human body 150. The control circuit 6 estimates the glucose concentration that corresponds to the output of the receiving module 5 based on the estimation information 13 stored in the memory 12.

With this configuration, the glucose concentration can be estimated by selecting, from the information table, the glucose concentration that corresponds to the output of the light receiving module 5. Therefore, the estimation can be performed by a simple process as compared with a configuration that conducts arithmetic processing with a learned model.

In the glucose concentration estimating device 1A of the second embodiment, the control circuit 6 obtains the attribution information from a plurality of test subjects. The learned model is a learned model in which the relationship between the reflected light information and the glucose concentration upon emitting the first to third light to the skin of every test subject is learned based on the information of the reflected light returning upon emitting the first to third light to the skin of the test subject concerned, and the actual measurement of the glucose concentration of the test subject concerned. The estimation information 13 is an information table generated from the learned model, and indicates the relationship among the attribute information of the test subject concerned, the reflected light information of the test subject that corresponds to the attribution information concerned, and the glucose concentration of the test subject that corresponds to the attribution information concerned. The control circuit 6 uses the information table to estimate the glucose concentration of the particular test subject from the attribute information of the particular test subject and the output of the light receiving module 5 for the particular test subject.

With this configuration, the glucose concentration of the subject to be estimated (target test subject) can be estimated by using an information table generated from a learned model for another test subject who has the same attribute as or a similar attribute to the target test subject. This makes it possible to easily and accurately estimate the glucose concentration of a target test subject having the same attribute as or a similar attribute to another test subject if an appropriate information table is prepared from an appropriate learned model for the above-mentioned “another test subject”.

### Relationship Between the Second Embodiment and the Claims

The control circuitry 6 and Step S202 in the second embodiment correspond to an estimating unit and an attribution information acquiring unit in the claims. The human body 150 corresponds to a living body in the claims. The memory 12 corresponds to a storage unit in the claims.

### Modifications

In the first embodiment, the four wavelengths are used. Specifically, one wavelength in the wavelength range of 1375 nm to 1395 nm, one wavelength in the wavelength range of 1575 nm to 1595 nm, one wavelength in the wavelength range of 1835 nm to 1855 nm, and one wavelength in the wavelength range of 2175 nm to 2255 nm are used. The first embodiment is not limited to this configuration, and a configuration in which five or more wavelengths are used from these wavelength ranges may be employed. Alternatively, a configuration in which one or more wavelengths in other wavelength ranges are added to the above-mentioned four wavelengths may be employed.

In the second embodiment, the three wavelengths are used. Specifically, one wavelength in the wavelength range of 1375 nm to 1395 nm, one wavelength in the wavelength range of 1575 nm to 1595 nm, and one wavelength in the wavelength range of 1835 nm to 1855 nm are used. The second embodiment is not limited to this configuration, and a configuration in which four or more wavelengths are used from these wavelength ranges may be employed. Alternatively, a configuration in which one or more wavelengths in other wavelength ranges are added to the above-mentioned three wavelengths may be employed.

In the above-described embodiments and the modifications thereof, a configuration has been described in which individual learned models are generated for the test subjects respectively, and attribute information is associated with individual information tables generated using the learned models, but the present disclosure is not limited to such configuration. For example, a learned model in which the relationship among the attribute information of the test subject, the information of the reflected light for the test subject, and the measured value of the glucose concentration of the test subject is learned may be generated. With this configuration, the relationship with the attribute is also learned. Thus, the glucose concentration corresponding to the attribute information of this test subject can be estimated by inputting the information of the reflected light of this test subject and the attribute information of this test subject into the learned model.

It will be apparent to those skilled in the art that various modifications and variations can be made in the present disclosure without departing from the spirit or scope of the present disclosure. Thus, it is intended that the present disclosure covers modifications and variations that come within the scope of the appended claims and their equivalents. In particular, it is explicitly contemplated that any part or whole of any two or more of the embodiments and their modifications described above can be combined and regarded within the scope of the present disclosure.

1. A glucose concentration estimating device comprising:
a first light source for irradiating a living body with a first light which includes any one wavelength in a wavelength range of 1375 nm to 1395 nm;
a second light source for irradiating the living body with a second light which includes any one wavelength in a wavelength range of 1575 nm to 1595 nm;
a third light source for irradiating the living body with a third light which includes any one wavelength in a wavelength range of 1835 nm to 1855 nm;
a light receiving element for receiving reflected light that returns from the living body upon irradiating the living body with the first light, the second light and the third light; and
an estimating unit that estimates a glucose concentration based on an output of the light receiving element.

2. The glucose concentration estimating device of claim 1 further comprising a fourth light source for irradiating the living body with a fourth light that includes one wavelength in a wavelength range of 2175 nm to 2255 nm, wherein the light receiving element is configured to be capable of receiving reflected light that returns from the living body upon irradiating the living body with the first light, the second light, the third light and the fourth light.

3. The glucose concentration estimating device of claim 1 further comprising a storage unit that stores estimation information, the estimation information including a learned model that is obtained by learning in advance a relationship between information on the reflected light and glucose concentration, wherein the estimating unit takes, as an estimated value of the glucose concentration, a glucose concentration that corresponds to the output of the light receiving element by using the estimation information stored in the storage unit.

4. The glucose concentration estimating device according to claim 3, wherein the learned model is a model in which learning is performed on a plurality of combinations of concentrations, and each of the plurality of combinations of concentrations includes a glucose concentration, a moisture concentration, and other components' concentration in a dermis layer set for an artificial skin.

5. The glucose concentration estimating device according to claim 3 further comprising an attribute information acquisition unit that acquires attribute information regarding an attribute of a test subject,
wherein the learned model is a model in which relationship among the attribution information of the test subject, information on the reflected light and the glucose concentration is learned based on the attribution information of the test subject, the information on the reflected light returning from a skin of the test subject and a measured value of the glucose concentration of the test subject, and
the estimating unit uses the estimation information to estimate the glucose concentration of the test subject from the attribute information of the test subject acquired by the attribute information acquisition unit and the output of the light receiving element corresponding to the test subject.

6. The glucose concentration estimating device according to claim 1 further includes a storage unit that stores estimation information, the estimation information including information that indicates a relationship between the information on the reflected light and the glucose concentration, and the estimation information being generated by using a learned model that is obtained by learning in advance a relationship between the information of the reflected light and the glucose concentration, wherein the estimating unit takes, as an estimated value of the glucose concentration, a glucose concentration that corresponds to the output of the light receiving element by using the estimation information stored in the storage unit.

7. The glucose concentration estimating device according to claim 6, wherein the learned model is a model in which learning is performed on a plurality of combinations of concentrations, and each of the plurality of combinations of concentrations includes a glucose concentration, a moisture concentration, and other components' concentration in a dermis layer set for an artificial skin.

8. The glucose concentration estimating device according to claim 6 further comprising an attribute information acquisition unit that acquires attribute information regarding an attribute of a test subject,
wherein the learned model is a model in which relationship between information on the reflected light and the glucose concentration is learned based on the information on the reflected light returning from a skin of the test subject and a measured value of the glucose concentration of the test subject for every said test subject,
the information that indicates a relationship between the information on the reflected light and the glucose concentration indicates relationship among attribute information of the test subject, the information on the reflected light of the test subject corresponding to the attribute information, and the glucose concentration of the test subject corresponding to the attribute information, and
the estimating unit uses the estimation information to estimate the glucose concentration of the test subject from the attribute information of the test subject acquired by the attribute information acquisition unit and the output of the light receiving element corresponding to the test subject.
